(19) United States
(12) Patent Application Publication
(10) Pub. No.: US 2004/0234478 A1
(43) Pub. Date: Nov. 25, 2004

(54) Personal care compositions containing a silicone elastomer

(75) Inventors: Mannie Lee Clapp, Mason, OH (US); Rebecca Ann Taylor, Cincinnati, OH (US); Brian Dale Barford, West Chester, OH (US); Jorge Max Sunkel, Cincinnati, OH (US); Karl Shiqing Wei, Mason, OH (US); Cheyne Pohlman Thomas, Independence, KY (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/841,181

(22) Filed: May 7, 2004

Related U.S. Application Data
(60) Provisional application No. 60/468,748, filed on May 8, 2003.
(60) Provisional application No. 60/469,554, filed on May 9, 2003.
(60) Provisional application No. 60/515,040, filed on Oct. 28, 2003.

(51) Int. Cl. A61K 7/06; A61K 7/11

(52) U.S. Cl. 424/70.12

(57) ABSTRACT

The present invention relates to a personal care composition containing a dispersed oil phase, a silicone elastomer and an aqueous phase. These compositions are rinsable and at the same time deposit oil and silicone elastomer onto all keratinous surfaces while providing improved skin feel. Additionally, the present invention relates to a method of using a rinse off personal care composition that contains an elastomer gel, a dispersion phase and an aqueous phase.

### CROSS REFERENCE TO RELATED APPLICATIONS

This application claims the benefit of Provisional application Ser. No. 60/468,748, filed May 8, 2003, of Provisional application Ser. No. 60/469,564, filed May 9, 2003, and of Provisional application Ser. No. 60/515,040, filed Oct. 28, 2003.

### TECHNICAL FIELD

The present invention relates to the field of personal care compositions for improving skin feel of keratinous surfaces. More specifically, rinsable personal care compositions are provided that provide excellent skin moisturization and conditioning.

### BACKGROUND OF THE INVENTION

Personal care compositions are well known and widely used. These compositions have long been employed to cleanse and moisturize skin, deliver actives, hide imperfections and to reduce the oiliness/shine associated with sebum

Skin conditioning compositions that provide moisturizing benefits are known. Many of these compositions are aqueous systems comprising an emulsified conditioning oil or other similar material stabilized with surfactant. Typically, skin moisturizing compositions are in the form of lotions meant to be applied to the skin after bathing and throughout the day if reapplication is necessary.

Skin is made up of several layers of cells, which coat and protect the keratin and collagen fibrous proteins that form the skeleton of its structure. The outermost of these layers, referred to as the stratum corneum, is known to be composed of 25 nm protein bundles surrounded by 8 nm thick layers. Anionic surfactants and organic solvents typically penetrate the stratum corneum membrane and, by delipidization (i.e. removal of the lipids from the stratum corneum), destroy its integrity. This destruction of the skin surface topography leads to a rough feel and may eventually permit the surfactant or solvent to interact with the keratin, creating irritation.

It is now recognized that maintaining the proper water gradient across the stratum corneum is important to its functionality. Most of this water, which is sometimes considered to be the stratum corneum's plasticizer, comes from inside the body. If the humidity is too low, such as in a cold climate, insufficient water remains in the outer layers of the stratum corneum to properly plasticize the tissue, and the skin begins to scale and becomes itchy. Skin permeability is also decreased somewhat when there is inadequate water across the stratum corneum. On the other hand, exposure to high water concentration for long periods of time on the outside of the skin causes the stratum corneum to ultimately sorb three to five times its own weight of bound water. This swells and puckers the skin and results in approximately a two to three fold increase in the permeability of the skin to water and moisturizer molecules. In the shower or bath, as skin becomes hydrated, this is recognized as an ideal time to deliver moisturizer to the skin since absorption of the moisturizer will be high.

It is further desirable to deliver the above skin conditioning benefits via an in-the-shower or in-the-bath lotion. Unfortunately, in the shower/bath, moisturizers are often readily rinsed from the skin. This is particularly true when surfactant is present.

Thus, a need exists for compositions, which will effectively deposit moisturizers and/or other skin benefit agents in the shower and/or bath and thereby assist the stratum corneum in maintaining its barrier and water-retention functions at optimum performance in spite of deleterious interactions which the skin may encounter in washing, work, and recreation. Desirable properties of such skin care compositions are to provide good skin feel.

Thus, it has now been found that rinsable personal care compositions that contain silicone elastomers and a dispersion oil phase will effectively co-deposit oils, elastomers and/or other skin benefit agents such in the shower and/or bath and provide softer skin feel across all skin types and at the same time assist the stratum corneum in maintaining its barrier and water-retention functions at optimum performance in spite of deleterious interactions which the skin may encounter in washing, work, and recreation and at the same time. Several publications have also disclosed the use of personal care compositions that contain silicone elastomers. See, e.g. U.S. Pat. No. 6,183,766. This publication teaches the use of silicone elastomers in combination with a lipid. However, the use of silicone elastomers is taught to degrease a lipid containing hand sanitizer product.

The present invention provides personal care compositions, which may further comprise skin benefit agents. These compositions provide improved skin appearance, aesthetics and skin feel during and/or after application, and are especially useful in providing improved deposition or effectiveness of skin conditioning agents to the desired area of the skin.

### SUMMARY OF THE INVENTION

The present invention relates to a personal care composition comprising, a dispersed oil phase, a silicone elastomer and an aqueous phase.

Inventors have found that silicone elastomers dispersed in a structured oil allows for co-depositing of oil and elastomer on the skin from rinsable compositions. The dispersion phase of oils prevents the silicone elastomer from aggregation in an aqueous environment. Surprisingly, the dispersed oil phase also serves as a carrier for these materials, which normally do not have a favorable deposition profile, and allows for appreciable deposition of these materials on skin. These compositions are rinsable and at the same time deposit oil and silicone elastomer onto all keratinous surfaces while providing improved skin feel.

### DETAILED DESCRIPTION OF THE INVENTION

All percentages and ratios used herein are by weight of the total composition and all measurements made are at 25° C., unless otherwise designated.

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

All publications cited herein are hereby incorporated by reference in their entirety.

The term “topical application”, as used herein, means to apply or spread the compositions of the present invention onto the surface of the skin.

The term “dermatologically-acceptable,” as used herein, means that the compositions or components thereof so described are suitable for use in contact with human skin without undue toxicity, incompatibility, instability, allergic response, and the like.

The term “gel-network” as used herein, means an emulsifying system comprised of fatty alcohol and a hydrophilic surfactant.

The term “safe and effective amount” as used herein means an amount of a compound, component, or composition sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound medical judgment.

Active and other ingredients useful herein may be categorized or described herein by their cosmetic and/or therapeutic benefit or their postulated mode of action. However, it is to be understood that the active and other ingredients useful herein can in some instances provide more than one cosmetic and/or therapeutic benefit or operate via more than one mode of action. Therefore, classifications herein are made for the sake of convenience and are not intended to limit an ingredient to the particularly stated application or applications listed.

The compositions of the invention are useful for topical application and for providing an essentially immediate (i.e., acute) skin feel following rinse off of the composition to on the keratinous surface. Without intending to be limited by theory, it is believed that this acute skin feel improvement results at least in part from therapeutic coverage or masking of skin imperfections by the deposition of oil and silicone elastomers.

More particularly, the compositions of the present invention are useful for regulating skin condition, including regulating visible and/or tactile discontinuities in skin, including but not limited to visible and/or tactile discontinuities in skin texture and/or color, more especially discontinuities associated with skin aging. Such discontinuities may be induced or caused by internal and/or external factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin.

Dispersed Oil Phase

The dispersed oil phase comprises a skin compatible oil. By definition, the dispersed phase will have negligible solubility in the external phase and will be present as discrete particles in the composition. The dispersed oil phase preferably comprises no more than about 80 weight percent of the composition, more preferably no more than about 70 weight percent, still more preferably no more than about 60 weight percent, and still more preferably no more than about 50 weight percent of the dispersed oil phase. The dispersed oil phase preferably comprises at least about 1 weight percent, more preferably at least about 5 weight percent, even more preferably at least about 7 weight percent, and still more preferably at least 10 weight percent of the composition.

The shear index is a measure of how shear thinning the materials are as described in the method described herein. When n=1, the materials are Newtonian, and while they may be viscous, they will generally not possess sufficient structure to entrap silicone elastomer gels and elastomer particles. Therefore it is preferred that the skin compatible oil be shear thinning either by virtue of its composition or the structurants that may be added. Preferably, the shear index of the dispersed oil phase will be less than about 0.9, more preferably less than about 0.75, even more preferably less than about 0.6, even more preferably less than about 0.45, and still more preferably less than about 0.3.

Skin Compatible Oils

A skin compatible oil is defined here, as an oil that is liquid or semi-solid at the temperature at which bathing is carried out that is deemed safe for use in cosmetics being either inert to the skin or actually beneficial. The most useful skin compatible oils for the present invention include ester oils, hydrocarbon oils, and silicone oils.

Ester oils, as the name implies, have at least one ester group in the molecule. One type of common ester oil useful in the present invention are the fatty acid mono and polyesters such as cetyl octanoate, octyl isonanoanate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate; sucrose ester and polyesters, sorbitol ester, and the like.

A second type of useful ester oil is predominantly comprised of triglycerides and modified triglycerides. These include vegetable oils such as jojoba, soybean, canola, sunflower, safflower, rice bran, avocado, almond, olive, sesame, persic, castor, coconut, and mink oils. Synthetic triglycerides can also be employed provided they are liquid at room temperature. Modified triglycerides include materials such as ethoxylated and maleated triglyceride derivatives provided they are liquids. Proprietary ester blends such as those sold by Finetex as Finsolv are also suitable, as is ethylhexanoic acid glyceride.

A third type of ester oil is liquid polyester formed from the reaction of a dicarboxylic acid and a diol. Examples of polyesters suitable for the present invention is the polyesters marketed by ExxonMobil under the trade name PURESYN ESTER.RTM.

A second class of skin compatible oils suitable for the present invention is liquid and semi-solid hydrocarbons. These include linear and branched oils such as liquid paraffin, squalene, squalane, mineral oil, low viscosity synthetic hydrocarbons such as polyalphaolefin sold by ExxonMobil under the trade name of PURESYN PAO and polybutene under the trade name PANALANE or INDOPOL. Light (low viscosity) highly branched hydrocarbon oils are also suitable.

Petrolatum is a unique hydrocarbon material and a useful component of the present invention. Its semi-solid nature can be controlled both in production and by the formulator through blending with other oils. Since it is only partially comprised of a liquid fraction at room temperature, it is more properly regarded as either the “structured oil phase” when present by itself or alternatively as the “structurant” when admixed with other skin compatible oils.

A third class of useful skin compatible oils is silicone based. They include linear and cyclic polydimethyl siloxane, organo functional silicones (alkyl and alkyl aryl), and amino silicones.

It is preferred that the dispersed oil phase will have some structure—i.e. it is not completely fluid. This can be accomplished through the use of structurants as is commonly done by those skilled in the art, or by the use of oils which are naturally structured, such as petrolatum. The structure serves to entrap the silicone elastomer in the dispersed oil phase droplet. In a preferred embodiment of the current invention, at least a portion of the silicone elastomer is entrapped in the dispersed oil phase after processing as observed via light microscopy.

Silicone Elastomers

Silicone elastomers are deformable, flexible cross-linked organopolysiloxane materials that are useful in the present invention and are illustrated in U.S. Pat. No. 5,654,362, herein incorporated by reference in its entirety. For the purposes of this invention, silicone elastomers are present in two forms: A silicone elastomer gel, in which the elastomer is combined with a compatible solvent, resulting in a homogeneous gel, and the dry elastomer polymer, which is usually supplied as a powder. When present in the claimed compositions, regardless of form (gelled or powder), the silicone elastomer preferably comprises no more than about 40 weight percent, more preferably no more than about 20 weight percent, and still more preferably no more than about 10 weight percent. When present, the silicone elastomer preferably comprises at least 0.5 weight percent.

Such elastomers can be used alone or with volatile or nonvolatile solvents. Examples of suitable solvents include, but are not limited to, volatile and non-volatile silicones, volatile and non-volatile alcohols, volatile and non-volatile esters, volatile and non-volatile hydrocarbons, and mixtures thereof. Preferred elastomer gels for use herein are elastomer/solvent blends having an elastomer to solvent ratio of from about 1:100 to about 1:1, more preferably from about 1:50 to about 1:5. Preferably the silicone elastomer gel has a viscosity of no more than 7,500,000 centipoise, more preferably no more than 500,000 centipoise. Preferably the silicone elastomer blend has a viscosity of at least than 1,000 centipoise, more preferably at least 10,000 centipoise. When present as a powder, the elastomer (prior to its addition to the skin compatible oil) is essentially ‘dry’

The elastomers are by definition crosslinked, the degree of which can be vary depending on the elastic properties of the polymer that are desired. Cross-linking materials may be hydrophilic (ethylene oxide and propylene oxide, for example), hydrophobic (dimethicone, vinyl dimethicone, alkyl, etc.) or combinations thereof.

Examples of suitable silicone elastomer gels include DC9040 (cyclomethicone and dimethicone crosspolymer blend), DC9010 and DC 9011 (Dow Corning.); (SFE 839 cyclomethicone and dimethicone/vinyldimethicone cross polymer blend, GE); (Gransil GCM (octamethylcyclotetrasiloxane and polysilicone-11 blend), KSG-21, KSG-210, KSG-310, KSG-320, KSG-330 and KSG-340, KSG-41, KSG-42, KSG-43, USG-103 all made by Shin-Etsu and varying in crosslinking agent and in solvent composition; and mixtures thereof.

Nonlimiting examples of silicone elastomer powders useful in the present invention are the KSP 100 and 200 series and the KMP series from Shin Etsu and DC9506 from Dow Corning and Toray.

When present, the silicone elastomer gels or powders preferably comprises no more than about 60 weight percent, more preferably no more than about 40 weight percent, and more preferably no more than about 20 weight percent. When present, the silicone elastomer preferably comprises at least 0.5 weight percent, more preferably at least 1 weight percent, and more preferably at least 2 weight percent.

In an embodiment of the current invention, the silicone elastomers are processed with the skin compatible oil in order to result in the entrapment of the elastomer by the skin compatible oil. Under these conditions, when the silicone elastomer is in the form of a silicone elastomer gel, there are two possible outcomes—the elastomer gel may be compatible with the skin compatible oil and a new gel would be formed, or the elastomer gel will not be compatible with the skin compatible oil and the elastomer would exist in separate domains within the dispersed skin compatible oil phase. A similar situation can occur when using a silicone elastomer powder, the powder may be compatible with the skin compatible oil and some gelling may occur, or the powder may be incompatible with the skin compatible oil and no gelling may occur. Preferred herein are the situations where the elastomer gel or elastomer powder is not compatible with the skin compatible oil and discrete domains of elastomer gel or elastomer powder are present within the dispersed oil phase. For elastomer gels, this can be predicted by using the Vaughn Solubility Parameter (VSP) (Christopher Vaughn, Cosmetics and Toiletries, Vol 103, 1988, pp 47-69) of the elastomer solvent as well as the skin compatible oil. Preferably, the solvent phase of the silicone elastomer gel has an absolute difference of at least 0.5 VSP units from the skin compatible oil, more preferably at least 0.8 VSP units, and even more preferably at least 1.2 VSP units. For example, for the blend of KSG-210 (elastomer in dimethicone) in petrolatum the difference is 1.41 (7.33 (petrolatum)-5.92(dimethicone)=1.41).

Preferred elastomer gels or powders for use herein are elastomer/skin compatible oil mixtures having an elastomer to skin compatible oil ratio of from about 1:100 to about 1:1, more preferably from about 1:50 to about 1:2, still more preferably from about 1:25 to about 1:3.

Aqueous Phase

The continuous aqueous phase of the present invention typically comprises from no more than about 90 weight percent of a fluid, preferably no more than 80%, even more preferably no more than about 70 weight percent, still more preferably no more than about 60 weight percent. The continuous aqueous phase of the present invention typically comprises at least 10 weight percent of a fluid, preferably at least 20 weight percent, even more preferably at least 30 weight percent, still more preferably at least 40 weight percent of a fluid. The term “fluid” as used herein means water, mono- and polyhydric alcohols (glycerin, propylene glycol, ethanol, isopropanol, sorbitol, etc.), or any material which is water miscible. A “continuous” phase is also described by those skilled in the art as an “external” phase.

Optional Ingredients

The compositions of the present invention may contain one or more additional skin care components. In a preferred embodiment, where the composition is to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment.

The CTFA Cosmetic Ingredient Handbook, Second Edition (1992) describes a wide variety of nonlimiting cosmetic and pharmaceutical ingredients commonly used in the personal care industry, which are suitable for use in the compositions of the present invention.

In any embodiment of the present invention, however, the additional components useful herein can be categorized by the benefit they provide or by their postulated mode of action. However, it is to be understood that the additional components useful herein can in some instances provide more than one benefit or operate via more than one mode of action. Therefore, classifications herein are made for the sake of convenience and are not intended to limit the active to that particular application or applications listed.

Structurants

The present invention may optionally comprise an oil structurant. The structurant can provide the dispersed phase with the correct rheological properties. This can aid in providing effective deposition and retention to the skin, the structured oil or oil phase should have a viscosity in the range of 100 to about 200,000 poise measured at 1 Sec-1, preferably 200 to about 100,000 poise, and most preferably 200 to about 50,000 poise as determined using the lipid rheology method described below. The amount of structurant required to produce this viscosity will vary depending on the oil and the structurant, but in general, the structurant will preferably be less than 75 weight percent of the dispersed oil phase, more preferably less than 50 weight percent, and still more preferably less than 35 weight percent of the dispersed oil phase.

The structurant can be either an organic or inorganic structurant. Examples of organic thickeners suitable for the invention are solid fatty acid esters, natural or modified fats, fatty acid, fatty amine, fatty alcohol, natural and synthetic waxes, and petrolatum, and the block copolymers sold under the name KRATON by Shell. Inorganic structuring agents include hydrophobically modified silica or hydrophobically modified clay. Nonlimiting examples of inorganic structurants are BENTONE 27V, BENTONE 38V or BENTONE GEL MIO V from Rheox; and CAB-O-SIL TS720 or CAB-O-SIL M5 from Cabot Corporation.

Structurants meeting the above requirements with the selected skin compatible oil can form 3-dimensional network to build up the viscosity of the selected oils. It has been found that such structured oil phases, i.e., built with the 3-dimensional network, are extremely desirable for use as wet-skin treatment compositions used in bathing. These structured oils can deposit and be retained very effectively on wet skin and retained after rinsing and drying to provide long-lasting after wash skin benefit without causing a too oily/greasy wet and dry feel. It is believed that the highly desirable in-use and after-use properties of such structured oils are due to their shear thinning Theological properties and the weak structure of the network. Due to its high low-shear viscosity, the 3-dimensional network structured oil can stick and retain well on the skin during application of the skin conditioner. After being deposited on the skin, the network yields easily during rubbing due to the weak structuring of the crystal network and its lower high-shear viscosity.

Surfactants

A wide variety of surfactants can be useful herein, both for emulsification of the dispersed phase as well as to provide acceptable spreading and in use properties for non-lathering systems. For cleansing applications, the surfactant phase also serves to clean the skin and provide an acceptable amount of lather for the user. The composition preferably contains no more than about 50 weight percent of a surfactant, more preferably no more than about 30 weight percent, still more preferably no more than about 15 weight percent, and even more preferably no more than about 5 weight percent of a surfactant. The composition preferably contains at least about 5 weight percent of a surfactant, more preferably at least about 3 weight percent, still more preferably at least about 1 weight percent, and even more preferably at least about 0.1 weight percent of a surfactant. For cleansing applications the personal care compositions preferably produces a Total Lather Volume of at least 300 ml, more preferably greater than 600 ml as described in the Lathering Volume Test. The personal care compositions preferably produces a Flash Lather Volume of at least 100 ml, preferably greater than 200 ml, more preferably greater than 300 ml as described in the Lathering Volume Test. The personal care compositions preferably produces a Flash Lather Volume of at least 100 ml, preferably greater than 200 ml, more preferably greater than 300 ml as described in the Lathering Volume Test.

Preferable surfactants include those selected from the group consisting of anionic surfactants, nonionic surfactants, amphoteric surfactants, non-lathering surfactants, emulsifiers and mixtures thereof. Non-limiting examples of surfactants useful in the compositions of the present invention are disclosed in U.S. Pat. No. 6,280,757, to McAtee et al., issued Aug. 28, 2001.

A) Anionic Surfactants

Non-limiting examples of anionic surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678, to Laughlin et al., issued Dec. 30, 1975.

A wide variety of anionic surfactants are useful herein. Non-limiting examples of anionic surfactants include those selected from the group consisting of sarcosinates, sulfates, isethionates, taurates, phosphates, lactylates, glutamates, and mixtures thereof. Amongst the isethionates, the alkoyl isethionates are preferred, and amongst the sulfates, the alkyl and alkyl ether sulfates are preferred.

Other anionic materials useful herein are fatty acid soaps (i.e., alkali metal salts, e.g., sodium or potassium salts) typically having from a fatty acid having about 8 to about 24 carbon atoms, preferably from about 10 to about 20 carbon atoms. These fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps and their preparation are described in detail in U.S. Pat. No. 4,557,853.

Other anionic materials include phosphates such as monoalkyl, dialkyl, and trialkylphosphate salts. Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Especially preferred for use herein is ammonium lauryl sulfate, ammonium laureth sulfate, sodium lauroyl sarcosinate, sodium cocoyl sarcosinate, sodium myristoyl sarcosinate, sodium lauroyl lactylate, and triethanolamine lauroyl lactylate.

B) Non-Ionic Surfactants

Non-limiting examples of nonionic surfactants for use in the compositions of the present invention are disclosed in McCutcheon's, Detergents and Emulsifiers, North American edition (1986), published by allured Publishing Corporation; and McCutcheon's, Functional Materials, North American Edition (1992).

Nonionic surfactants useful herein include those selected from the group consisting of alkyl glucosides, alkyl polyglucosides, polyhydroxy fatty acid amides, alkoxylated fatty acid esters, sucrose esters, amine oxides, and mixtures thereof.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, lauramine oxide, cocoamine oxide and mixtures thereof.

C) Amphoteric Surfactants

The term “amphoteric surfactant,” as used herein, is also intended to encompass zwitterionic surfactants, which are well known to formulators skilled in the art as a subset of amphoteric surfactants.

A wide variety of amphoteric lathering surfactants can be used in the compositions of the present invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Non-limiting examples of amphoteric surfactants useful in the compositions of the present invention are disclosed in McCutcheon's, Detergents and Emulsifiers, North American edition (1986), published by allured Publishing Corporation; and McCutcheon's, Functional Materials, North American Edition (1992).

Non-limiting examples zwitterionic surfactants are those selected from the group consisting of betaines, sultaines, hydroxysultaines, alkyliminoacetates, iminodialkanoates, aminoalkanoates, and mixtures thereof.

Preferred surfactants for use herein are the following, wherein the anionic surfactant is selected from the group consisting of ammonium lauroyl sarcosinate, sodium trideceth sulfate, sodium lauroyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isetlionate, sodium cetyl sulfate, sodium lauroyl lactylate, triethanolamine lauroyl lactylate, and mixtures thereof, wherein the non-ionic surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

D) Non-Lathering Surfactants

A wide variety of non-lathering surfactants are useful herein. The composition of the present invention can comprise a sufficient amount of one or more non-lathering surfactants to emulsify the dispersed phase to yield an appropriate particle size and good application properties on wet skin.

Nonlimiting examples of these non-lathering compositions are: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

E) Emulsifier systems

In addition, there are several commercial emulsifier mixtures that are useful in some embodiments. Examples include PROLIPID 141 (glyceryl stearate, behenyl alcohol, palmitic acid, stearic acid, lecithin, lauryl alcohol, myristyl alcohol and cetyl alcohol) and 151 (Glyceryl stearate, cetearyl alcohol, stearic acid, 1-propanamium, 3-amino-N-(2-(hydroxyethyl)-N-N-Dimethyl,N-C(16-18) Acyl Derivatives, Chlorides) from ISP; POLAWAX NF (Emulsifying wax NF), INCROQUAT BEHENYL TMS (behentrimonium sulfate and cetearyl alcohol) from Croda; and EMULLIUM DELTA (cetyl alcohol, glyceryl stearate, peg-75 stearate, ceteth-20 and steareth-20) from Gattefosse.

Thickening/Aqueous Phase Stability Agents

The compositions of the present invention, in some embodiments, may further include one or more thickening/aqueous phase stability agents. Because different stability agents thicken with different efficiencies, it is difficult to provide an accurate compositional range, however, when present, the composition preferably comprises no more than about 10 weight percent, more preferably no more than about 8 weight percent, and still more preferably no more than about 7 weight percent of the personal care composition. When present, the thickening/aqueous phase stability agent preferably comprises at least about 0.01 weight percent, more preferably at least about 0.05 weight percent, and still more preferably at least about 0.1 weight percent of the personal care composition. A better method of describing the Stability Agent is to say that it must build viscosity in the product. This can be measured using the Stability Agent Viscosity Test. Preferably, the stability agent produces a viscosity in this test of at least 1000 cps, more preferably at least 1500 cps, and still more preferably at least 2000 cps.

Nonlimiting examples of thickening agents useful herein include carboxylic acid polymers such as the carbomers (such as those commercially available under the tradename CARBOPOL® 900 series from B.F. Goodrich; e.g., CARBOPOL® 954). Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, PEMULEN TR-1, and PEMULEN TR-2, from B.F. Goodrich.

Other nonlimiting examples of thickening agents include crosslinked polyacrylate polymers including both cationic and nonionic polymers.

Still other nonlimiting examples of thickening agents include the polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

Another nonlimiting class of thickening agents useful herein is the polysaccharides. Nonlimiting examples of polysaccharide gelling agents include those selected from cellulose, and cellulose derivatives. Preferred among the alkyl hydroxyalkyl cellulose ethers is the material given the CTFA designation cetyl hydroxyethylcellulose, which is the ether of cetyl alcohol and hydroxyethylcellulose, sold under the tradename NATROSEL® CS PLUS from Aqualon Corporation (Wilmington, Del.). Other useful polysaccharides include scleroglucans which are a linear chain of (1-3) linked glucose units with a (1-6) linked glucose every three units, a commercially available example of which is CLEAROGEL™ CS11 from Michel Mercier Products Inc. (Mountainside, N.J.).

Another nonlimiting class of thickening agents useful herein are the gums. Nonlimiting examples of gums useful herein include hectorite, hydrated silica, xantham gum, and mixtures thereof.

Yet another nonlimiting class of thickening agents useful herein are the modified starches. Acrylate modified starches such as WATERLOCK® from Grain Processing Corporation may be used. Hydroxypropyl starch phosphate, tradename STRUCTURE XL from National Starch is another example of a useful modified starch, and other useful examples include ARISTOFLEX HMB (Ammonium Acrylodimethyltaruate/Beheneth-25 Methacrylate Crosspolymer) from Clariant.

Cationic Polymers

The present invention may also contain organic cationic deposition polymer Concentrations of the cationic deposition polymer preferably range from about 0.025% to about 3%, more preferably from about 0.05% to about 2%, even more preferably from about 0.1% to about 1%, by weight of the personal care composition.

Suitable cationic deposition polymers for use in the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the personal cleansing composition. The average molecular weight of the cationic deposition polymer is between about 5,000 to about 10 million, preferably at least about 100,000, more preferably at least about 200,000, but preferably not more than about 2 million, more preferably not more than about 1.5 million. The polymers also have a cationic charge density ranging from about 0.2 meq/gm to about 5 meq/gm, preferably at least about 0.4 meq/gm, more preferably at least about 0.6 meq/gm., at the pH of intended use of the personal cleansing composition, which pH will generally range from about pH 4 to about pH 9, preferably between about pH 5 and about pH 8.

Nonlimiting examples of cationic deposition polymers for use in the personal care composition include polysaccharide polymers, such as cationic cellulose derivatives. Preferred cationic cellulose polymers are the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers with the most preferred being KG-30M.

Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Aqualon.

Other suitable cationic deposition polymers include synthetic cationic polymers. The cationic polymers suitable for use in the cleansing composition herein are water soluble or dispersible, non crosslinked, cationic polymers having a cationic charge density of from about 4 meq/gm to about 7 meq/gm, preferably from about 4 meq/gm to about 6 meq/gm, more preferably from about 4.2 meq/gm to about 5.5 meq/gm. The select polymers also must have an average molecular weight of from about 1,000 to about 1 million, preferably from about 10,000 to about 500,000, more preferably from about 75,000 to about 250,000.

The concentration of the cationic polymerin the personal care composition ranges from about 0.025% to about 5%, preferably from about 0.1% to about 3%, more preferably from about 0.2% to about 1%, by weight of the composition.

A non limiting example of a commercially available synthetic cationic polymer for use in the cleansing compositions is polymethyacrylamidopropyl trimonium chloride, available under the trade name POLYCARE 133, from Rhodia, Cranberry, N.J., U.S.A.

Shiny Particles

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®.

A second class of interference pigment is based on cholesteric liquid crystal, e.g. HELICONE® HC supplied by KOBO products. HELICONE® HC is composed of transparent platelets of polyacrylates with a helical superstructure. As part of this structure, cigar-shaped liquid crystal molecules are fixed into layers of parallel rows. Each layer has a slightly different molecular orientation and the distance between two layers with the same molecular orientation defines as the “pitch”, which determines the color. This type pigment is hydrophobic. Therefore, they can be used without surface treatment

Other Optional Ingredients

Other non limiting examples of optional ingredients include benefit agents that are selected from the group consisting of vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol) and antibacterial agents and mixtures thereof. These materials can be used at ranges sufficient to provide the required benefit, as would be obvious to one skilled in the art.

Adhesive materials, such as cross-linked silicone resin-dimethicone blends, as well as acrylate based adhesives and other commonly used film-forming materials such as the modified PVP polymers sold under the tradename GANEX from ISP may also be used in the present invention.

Analytical Methods

Lipid Rheology Test

Lipid rheology is measured on a TA Instruments AR2000 stress-controlled rheometer with a Peltier temperature controlled sample stage or an equivalent. A parallel plate geometry is used with a 40 mm plate and a 1 mm gap. The lower plate is heated to 85° C. and the melted lipid and structurant (if present) is added onto the lower plate and allowed to equilibrate. The upper plate is then lowered to the 1 mm gap while ensuring the lipid fills the gap fully, [spinning the top plate and adding more lipid to promote wicking], and the sample is cooled quickly to 25° C. and equilibrated at 25° C. for 5 minutes. Viscosity is then measured using a stress-ramp procedure common on these types of machines using a logarithmic stress ramp from 20 to 2000 Pa at a rate of 60 seconds per decade [2 minute ramp test], with 20 measurements points per decade. The starting and ending stress is sufficient to induce flow and reach a shear rate of at least 10 sec-1. Viscosity is recorded and the data fitted to a power law model using Equation 1. Only points between 0.001 sec-1 and 40 seconds-1 are to be used in the power law fit. The viscosity at 1.0 sec-1 is calculated from Equation 1. One should carefully watch the sample during the test so that when the material is ejected from under the plate, the method is stopped.

Viscosities are recorded and the data fit to a power law with the following Equation 1:

η=κ·γ(dot)(ⁿ⁻¹⁾

where η=viscosity, κ is the consistency and γ(dot) is the shear rate, and n is the shear index.

The viscosity at 1 sec-I is then calculated using the calculated values of κ and n from the fitted

Lather Volume

Lather volume of a liquid personal cleansing composition is measured using a graduated cylinder and a tumbling apparatus. A 1,000 ml graduated cylinder is chosen which is marked in 10 ml increments and has a height of 14.5 inches at the 1,000 ml mark from the inside of its base (for example, Pyrex No. 2982). Distilled water (100 grams at 23° C.) is added to the graduated cylinder. The cylinder is clamped in a rotating device, which clamps the cylinder with an axis of rotation that transects the center of the graduated cylinder. One gram of the total personal cleansing composition (0.5 g of the cleansing phase and 0.5 g of the benefit phase) is added into the graduated cylinder and the cylinder is capped. The cylinder is rotated at a rate of 10 revolutions in about 20 seconds, and stopped in a vertical position to complete the first rotation sequence. A timer is set to allow 30 seconds for the lather thus generated to drain. After 30 seconds of such drainage, the first lather volume is measured to the nearest 10 ml mark by recording the lather height in ml up from the base (including any water that has drained to the bottom on top of which the lather is floating).

If the top surface of the lather is uneven, the lowest height at which it is possible to see halfway across the graduated cylinder is the first lather volume (ml). If the lather is so coarse that a single or only a few foam cells (“bubbles”) reach across the entire cylinder, the height at which at least 10 foam cells are required to fill the space is the first lather volume, also in ml up from the base. Foam cells larger than one inch in any dimension, no matter where they occur, are designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum foam height is 1,000 ml (even if the total foam height exceeds the 1,000 ml mark on the graduated cylinder). One minute after the first rotation is completed, a second rotation sequence is commenced which is identical in speed and duration to the first rotation sequence. The second lather volume is recorded in the same manner as the first, after the same 30 seconds of drainage time. A third sequence is completed and the third lather volume is measured in the same manner, with the same pause between each for drainage and taking the measurement.

The lather result after each sequence is added together and the Total Lather Volume determined as the sum of the three measurements, in ml. The Flash Lather Volume is the result after the first rotation sequence only, in ml, i.e., the first lather volume. Compositions according to the present invention perform significantly better in this test than similar compositions in conventional emulsion form.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total cleansing, treatment compositions, unless otherwise specified.

| | Ex. 1 | Ex. 2 | Ex. 3 | Ex. 4 | Ex. 5 | Ex. 7 | Ex. 8 | Ex. 9 |
|---|---|---|---|---|---|---|---|---|
| Ingredient | wt % | wt % | wt % | wt % | wt % | wt % | wt % | wt % |
| I. Aqueous Phase Composition | | | | | | | | |
| Hydroxypropyl Starch | 3.5 | 4.0 | 3.5 | 3.5 | 3.5 | 3.5 | 3.5 | 4 |
| Phosphate (Structure XL from | | | | | | | | |
| National Starch) | | | | | | | | |
| Emulsifying Wax NF | | 3.0 | 2.75 | 2.75 | 2.75 | | | 3.0 |
| (Polawax from Croda) | | | | | | | | |
| Behenetrimonium | 2.25 | | | | | 2.0 | 2.0 | |
| methosulfate and cetearyl | | | | | | | | |
| alcohol (Incroquat Behenyl | | | | | | | | |
| TMS from Croda) | | | | | | | | |
| Fragrance | 1.0 | 1.0 | 1.0 | 1.0 | 1.0 | 1.0 | 1.0 | 1.0 |
| Preservatives | 0.8 | 0.8 | 0.8 | 0.8 | 0.8 | 0.8 | 0.8 | 0.8 |
| Water | Q.S. | Q.S. | Q.S. | Q.S. | Q.S. | Q.S. | Q.S. | Q.S. |
| II. Lipid/Elastomer phase | | | | | | | | |
| Composition | | | | | | | | |
| Petrolatum (Superwhite | 20 | 15 | 25 | 20 | 10 | 15 | 15 | |
| Protopet from WITCO) | | | | | | | | |
| Gelled Mineral Oil (Versagel | | | | | | | | 12 |
| M750 from Penreco) | | | | | | | | |
| Mineral Oil (Hydrobrite 1000 | 5 | | | | | | | |
| PO White MO from WITCO) | | | | | | | | |
| Jojoba Oil (Lipovol J from | | | 5 | | | | | |
| Lipo) | | | | | | | | |
| Dimethicone copolyol | | | 2.0 | 5.0 | | | | |
| crosspolymer (KSG 210 from | | | | | | | | |
| Shin-Etsu) | | | | | | | | |
| Cyclomethicone and Vinyl | | | | | | | | 3 |
| Dimethicone/Dimethicone | | | | | | | | |
| Crosspolymer (Shin-Etsu | | | | | | | | |
| USG-103) | | | | | | | | |
| Cyclomethicone and | | | | | 3.5 | | | |
| Dimethicone Crosspolymer | | | | | | | | |
| (Dow Corning 9040) | | | | | | | | |
| Vinyl Dimethicone/Lauryl | 5 | | | | | | | |
| Dimethicone Crosspolymer, | | | | | | | | |
| Mineral Oil (KSG-41 from | | | | | | | | |
| Shin-Etsu) | | | | | | | | |
| Cyclopentasiloxane and PEG- | | 3.0 | | | | | | |
| 12 Dimethicone | | | | | | | | |
| Crosspolymer (Dow Corning | | | | | | | | |
| 9010) | | | | | | | | |
| Vinyl | | | | | | 7.0 | | |
| Dimethicone/Methicone | | | | | | | | |
| Silsesquioxane Crosspolyer | | | | | | | | |
| (KSP-105 from Shin-Etsu) | | | | | | | | |
| Dimethicone/Vinyl | | | | | | | 3.5 | |
| Dimethicone Crosspolymer | | | | | | | | |
| and C12-14 Pareth-12 (Dow | | | | | | | | |
| Corning 9506) | | | | | | | | |

The personal care composition of Example 1-9 can be prepared by conventional formulation and mixing techniques.

Prepare the aqueous phase composition by first dispersing the hydroxypropyl phosphate in water. Add and heat the emulsifying wax to 160 F. Next, place the mixing vessel in a water bath to cool to under 100 F. Add fragrance

Prepare the lipid phase by first premixing if multiple lipids are present. Then, add the elastomers and the lipid to the premix at 160 F. Then, mill the elastomer phase and the lipid phase under high shear using a Silverson L4R High Shear Mixer or equivalent until the dispersion is smooth and no large agglomerates of elastomer are present. Then, quickly add the still fluid lipid/Elastomer phase to the aqueous phase (<80F) with increased agitation. Add preservatives and agitate until product is smooth.

### Example 10-12

| | Ex. 10 | Ex. 11 | Ex. 12 |
|---|---|---|---|
| Ingredient | wt % | wt % | wt % |
| I. Cleansing Phase Composition | | | |
| Miracare SLB-365 (from Rhodia) | 47.4 | 47.4 | 47.4 |
| (Sodium Trideceth Sulfate, Sodium | | | |
| Lauramphoacetate, Cocamide MEA) | | | |
| Guar Hydroxypropyltrimonium Chloride | 0.7 | 0.7 | 0.7 |
| (N-Hance 3196 from Aqualon) | | | |
| PEG 90 M (Polyox WSR 301 from | 0.2 | 0.2 | 0.2 |
| Dow Chemical) | | | |
| Sodium Chloride | 3.5 | 3.5 | 3.5 |
| Preservatives | 0.84 | 0.84 | 0.84 |
| Citric Acid | 0.4 | 0.4 | 0.4 |
| Perfume | 2.0 | 2.0 | 2.0 |
| Expancel 091 DE 40 d30 (from | 0.4 | 0.4 | 0.4 |
| Expancel, Inc.) | | | |
| Water | Q.S. | Q.S. | Q.S. |
| (pH) | (6.0) | (6.0) | (6.0) |
| II. Lipid phase Composition | | | |
| Petrolatum (Superwhite Protopet | 73.08 | 72.08 | 72.08 |
| from WITCO) | | | |
| Mineral Oil (Hydrobrite 1000 PO | 23.92 | 23.92 | 23.92 |
| White MO from WITCO) | | | |
| Dimethicone copolyol crosspolymer | 3.0 | | |
| (KSG 210 from Shin-Etsu) | | | |
| Cyclomethicone and | | 4.0 | |
| Dimethicone Crosspolymer | | | |
| (Dow Corning 9040) | | | |
| Vinyl Dimethicone/Methyl Silsesquioxane | | | 4 |
| Crosspolymer (KSP 101 from Shin Etsu) | | | |

The composition described above can be prepared by conventional formulation and mixing techniques. The cleansing phase composition is prepared by first adding citric acid into water at a 1:3 ratio to form a citric acid premix. The following ingredients are then added into the main mixing vessel in the following sequence: water, Miracare SLB-354, sodium chloride, and preservatives. Start agitation of the main mixing vessel. In a separate mixing vessel, disperse polymer (N-Hance 3196) in water at 1:10 ratio to form a polymer premix. Add the completely dispersed polymer premix into the main mixing vessel with continuous agitation. Disperse PEG 90M (Polyox WSR 301) in water and then add to the main mixing vessel. Then, add the rest of the water, perfume, and Expancel into the batch. Keep agitation until a homogenous solution forms.

The lipid phase is prepared by adding petrolatum into a mixing vessel. Heat the vessel to 190° F. Then add mineral oil with agitation. The Elastomer is milled into the lipids at 160 F and then allow the vessel to cool down with slow agitation.

The cleansing and lipid phases are density matched to within 0.05 g/cm³. Both phases are packaged into a single container using conventional toothpaste-tube filler equipment. The sample stage spins the bottle during filling process to create a striped appearance. The stripe size is about 6 mm in width and 100 mm in length.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A personal care composition comprising,
a) a silicone elastomer gel;
b) a dispersed oil phase; and
c) an aqueous phase;
wherein said dispersed oil phase is present at a greater percentage by weight than said silicone elastomer;
wherein said silicone elastomer gel is entrapped within said dispersed oil phase.

2. The composition of claim 1, wherein said silicone elastomer comprises no more than about 40 weight percent of said composition.

3. The composition of claim 1, wherein said silicone elastomer comprises at least 1 weight percent of said composition.

4. The composition of claim 1, wherein the dispersed oil phase has a shear index of less than 0.9

5. The composition of claim 1 wherein the dispersed oil phase has a shear index of less than 0.6.

6. The composition of claim 1 wherein the dispersed oil phase comprises a skin compatible oil phase.

7. The composition of claim 6, wherein the ratio of said silicone elastomer to said skin compatible oil phase is from about 1:100 to about 1:1.

8. The composition of claim 1, wherein said silicone elastomer gel has a solvent phase, said solvent phase having an absolute difference of at least 1.2 Vaughn Solubility Parameter units.

9. The composition of claim 1, wherein said dispersed oil phase comprises no more than about 80 weight percent of said composition.

10. The composition of claim 1, wherein said dispersed oil phase is selected from the group consisting of petrolatum, mineral oil, silicone, triglycerides, esters and mixtures thereof.

11. The composition of claim 1, wherein said aqueous phase is water.

12. The composition of claim 1, which additionally comprises a surfactant.

13. The composition of claim 12, wherein the composition comprises no more than about 5 weight percent of said surfactant.

14. The composition of claim 12, which additionally comprises a polymer.

15. The composition of claim 12, wherein the surfactant is present in a gel-network.

16. The composition of claim 1, wherein the composition comprises at least 5 weight percent of a surfactant.

17. The composition of claim 16, wherein said composition comprises at least one anionic surfactant.

18. The composition of claim 1, further comprising one or more benefit agents selected from the group consisting of vitamins, sunscreens, thickening agents, preservatives, anti-acne medicaments, antioxidants, skin soothing and healing agents, chelators and sequestrants, fragrances, essential oils, skin sensates, pigments, pearlescent agents, lakes, colorings, anti-bacterial agents and mixtures thereof.

19. A method of using a personal care composition comprising:
a) applying to a substrate a personal care composition comprising;
i. a silicone elastomer gel;
ii. a dispersed oil phase; and
iii. an aqueous phase;
wherein said dispersed oil phase is present at a greater percentage by weight than said silicone elastomer; and
b) rinsing off said personal care composition.

20. The method of claim 19 wherein said silicone elastomer gel is entrapped within said dispersed oil phase.
